(19) United States
(12) Patent Application Publication
(10) Pub. No.: US 2019/0221283 A1
(43) Pub. Date: Jul. 18, 2019

(54) APPARATUS AND METHOD FOR TRANSCRIPTOME ANALYSIS

(71) Applicants: Satoshi KONDO, Miyoshi-shi (JP); Chikara OHTO, Toyota-shi (JP); Madoka ABE, Nagoya-shi (JP); Naohiro AOKI, Tokyo (JP); Akari FUKUDA, Joetsu-shi (JP); Tatsuro HIROSE, Joetsu-shi (JP); Atsushi NAGANO, Otsu-shi (JP)

(72) Inventors: Satoshi KONDO, Miyoshi-shi (JP); Chikara OHTO, Toyota-shi (JP); Madoka ABE, Nagoya-shi (JP); Naohiro AOKI, Tokyo (JP); Akari FUKUDA, Joetsu-shi (JP); Tatsuro HIROSE, Joetsu-shi (JP); Atsushi NAGANO, Otsu-shi (JP)

(73) Assignees: TOYOTA JIDOSHA KABUSHIKI KAISHA, Toyota-shi (JP); THE UNIVERSITY OF TOKYO, Tokyo (JP); National Agriculture and Food Research Organization, Tsukuba-shi (JP); RYUKOKU UNIVERSITY, Kyoto-shi (JP)

(21) Appl. No.: 16/244,459

(22) Filed: Jan. 10, 2019

(30) Foreign Application Priority Data
Jan. 12, 2018 (JP) 2018-003697

(51) Int. Cl. G16B 20/00

(52) U.S. Cl. CPC G16B 20/00

(57) ABSTRACT

With the use of nucleotide sequence data concerning transcription products, transcriptome analysis is performed with higher precision. Concerning a plurality of data sets including the objective variable data and the gene expression level data, a plurality of subdata sets are generated by randomly deleting the gene expression level data, and the method of regularization is applied to the plurality of subdata sets to calculate relevant estimation formulae and then generate the lists of genes included in the estimation formula.

### CROSS REFERENCE TO RELATED APPLICATIONS

The present application claims priority from Japanese patent application JP 2018-3697 filed on Jan. 12, 2018, the content of which is hereby incorporated by reference into this application.

### BACKGROUND

### Technical Field

The present disclosure relates to an apparatus and a method for transcriptome analysis comprising analyzing information concerning a transcriptome.

### Background Art

As an attempt of estimating an organism's phenotype based on gene expression, a method of multiple regression analysis using gene expression data and phenotype data is known (Nature Communications 5, Article number: 5792, 2014; and WO 2016/148107). In the method disclosed in Nature Communications 5, Article number: 5792, 2014, limited gene expression data were used by, for example, selectively adopting data of the same operon exhibiting the highest expression level so as to avoid overlapping of gene expression data.

In general, the term “transcriptome” refers to all transcription products existing in tissue or cells under given conditions. Transcriptomes encompasses a transcription product from a coding region of the genome (i.e., mRNA) and a transcription product from a non-coding region of the genome (i.e., ncRNA). Through transcriptome analysis, a novel finding based on a state of gene expression, such as variation in gene expression affected by environmental factors or identification of genes expressed associated with phenotypes, can be achieved.

When analyzing a transcriptome, for example, transcription products existing in tissue or cells are extensively assayed with the use of microarray techniques or next-generation sequencing techniques. The assayed data are large quantity of nucleotide sequence data, which are typical big data.

As a method of statistical analysis of the data obtained, as disclosed in JP Patent No. 5854346, a method of adopting a technique of principal component analysis, which is one of technique of multivariate analyses, is known. Such method comprises conducting main component analysis concerning the training data instead of the nucleotide sequence data obtained by analysis, to achieve results that are comparable among samples under different conditions.

As a method of transcriptome analysis, as disclosed in JP 2017-51118 A, a method of generating the property variable estimation model for a target of analysis based on gene expression information (condition variable) and trait information (property variable) is known. In the method disclosed in JP 2017-51118 A, a regression model having regularization terms is generated by designating a property variable as an objective variable (i.e., a dependent variable) and each of condition variables as an explanatory variable. As a formula for determining a regression model, the least absolute shrinkage and selection operator regression (LASSO) method is exemplified.

LASSO regression is a technique of regularization (L1 regularization) adopted for prevention of overfitting in the fields of statistics, machine learning, and the like. LASSO regression is a technique of regression modeling based on sparse regularization, which is carried out by designating the parameter of less important data selected from a large quantity of data as zero and removing the same from the data (Robert Tibshirani, Journal of the Royal Statistical Society, Series B (Methodological) Vol. 58, No. 1, 1996, pp. 267-288).

### SUMMARY

In the transcriptome analysis described above, the number of transcription products with the available nucleotide sequences is very large relative to the number of samples to be analyzed. By the method disclosed in Nature Communications 5, Article number: 5792, 2014, accordingly, it was difficult to attain the meaningful results of analysis. In the case of the method of analysis that adopts the LASSO regression analysis disclosed in JP 2017-51118 A, satisfactory results of analysis are expected even if the number of transcription products with the available nucleotide sequences is very large relative to the number of samples to be analyzed. However, transcriptome analysis was required to show further improvement in accuracy of the results of analysis.

Under the above circumstances, the present disclosure provides an apparatus and a method for transcriptome analysis that enable more accurate transcriptome analysis with the use of the nucleotide sequence data concerning a transcription product.

The present disclosure encompasses the following.
(1) An apparatus for transcriptome analysis comprising:

a data set-generator that generates the first to the mth subdata sets (m≥2) by randomly deleting gene expression level data from a plurality of data sets including objective variable data and gene expression level data;

an estimation formula-calculator that applies the method of regression analysis having regularization terms to each of the first to the mth subdata sets to calculate the first to the mth estimation formulae using objective variable data as objective variables and gene expression level data as explanatory variables; and

a list of genes-generator that generates a list of genes corresponding to the gene expression level data included in the first to the mth estimation formulae.
(2) The apparatus for transcriptome analysis according to (1), wherein the estimation formula-calculator adopts the least absolute shrinkage and selection operator (LASSO) method as the method of regression analysis.
(3) The apparatus for transcriptome analysis according to (1), wherein the data set-generator generates 1,000 to 20,000 types of subdata sets (m=1,000 to 20,000).
(4) The apparatus for transcriptome analysis according to (1), wherein the list of genes-generator calculates the appearance probabilities of genes based on the results of the first to the mth estimation formulae and generates a list of genes in connection with the calculated appearance probabilities.
(5) The apparatus for transcriptome analysis according to (1), wherein the list of genes-generator retrieves the annotation information of the genes included in the list from the database storing the annotation information of the genes and generates a list of genes in connection with the retrieved annotation information.
(6) The apparatus for transcriptome analysis according to (1), which further comprises a estimation model formulae-generator that generates estimation model formulae concerning given objective variables by subjecting a plurality of genes included in the list generated by the list of genes-generator to multiple regression analysis using the objective variable data and the gene expression level data included in the plurality of data sets.
(7) A method of transcriptome analysis comprising:

a step of generating a subdata set that generates a subdata set by randomly deleting gene expression level data from a plurality of data sets including objective variable data and gene expression level data;

a step of calculating an estimation formula that applies the method of regularization to the subdata set to calculate an estimation formula using objective variable data as objective variables and gene expression level data as explanatory variables;

a step of gene recording that records genes corresponding to the gene expression level data included in the estimation formula; and

a step of generating a list of genes that repeats the step of generating the subdata set, the step of calculating an estimation formula and the step of gene recording m number of times (m≥2) to generate a list of the recorded genes.
(8) The method of transcriptome analysis according to (7), wherein the step of calculating an estimation formula adopts the least absolute shrinkage and selection operator (LASSO) method as the method of regularization.
(9) The method of transcriptome analysis according to (7), wherein the step of generating a subdata set generates 1,000 to 20,000 types of subdata sets (m=1,000 to 20,000).
(10) The method of transcriptome analysis according to (7), wherein the step of generating a list of genes calculates the appearance probabilities of genes based on the results of the first to the mth estimation formulae generated through repetition of 1 to m number of times and generates a list of genes in connection with the calculated appearance probability.
(11) The method of transcriptome analysis according to (7), wherein the step of generating a list of genes retrieves the annotation information of the genes included in the list from the database storing the annotation information of the genes and generates a list of genes in connection with the retrieved annotation information.
(12) The method of transcriptome analysis according to (7), which further comprises, following the step of generating a list of genes, a step of generating an estimation model formula that generates an estimation model formula concerning given objective variables by subjecting a plurality of genes included in the generated list to multiple regression analysis using the objective variable data and the gene expression level data included in the plurality of data sets.

### Advantageous Effects

The apparatus and the method for transcriptome analysis according to the present disclosure enable transcriptome analysis with high accuracy. With the use of the apparatus and the method for transcriptome analysis according to the present disclosure, accordingly, analysis of variations in gene expression caused by particular factor such as a given state or condition, expression analysis of genes correlated with phenotypes, prediction analysis of traits based on gene expression, and the like can be performed with high accuracy.

### BRIEF DESCRIPTION OF THE DRAWINGS

FIG. 1 shows a functional block diagram demonstrating an embodiment of the apparatus for transcriptome analysis of the present disclosure.

FIG. 2 shows a flow chart demonstrating an embodiment of the method for transcriptome analysis of the present disclosure.

FIG. 3 shows a characteristic diagram demonstrating an example of a list of genes outputted by the apparatus and the method for transcriptome analysis.

FIG. 4 shows a characteristic diagram demonstrating another example of a list of genes outputted by the apparatus and the method for transcriptome analysis.

FIG. 5 shows a functional block diagram demonstrating another embodiment of the apparatus for transcriptome analysis of the present disclosure.

FIG. 6 shows a flow chart demonstrating another embodiment of the method for transcriptome analysis of the present disclosure.

FIG. 7 shows a characteristic diagram demonstrating the correlation between estimated values outputted by the apparatus and the method for transcriptome analysis and measured values.

FIG. 8 shows a block diagram demonstrating a constitution of the predictive-evaluation system according to the present disclosure.

FIG. 9 shows a photograph of Arroz da Terra and Ouu365 taken 14 days after germination.

FIG. 10 shows a characteristic diagram demonstrating the results of QTL analysis of the dry weight of the terrestrial part of the BIL104 line.

FIG. 11 shows a characteristic diagram demonstrating a relationship between the number of lines prepared in the examples and the dry weight of the terrestrial part.

FIG. 12 shows a characteristic diagram demonstrating the fresh weight of the terrestrial part of the lines prepared in the examples.

FIG. 13-1 shows a characteristic diagram demonstrating a list of 158 genes selected as biomarkers for the expression level at high frequency in the examples.

FIG. 13-2 shows a characteristic diagram demonstrating a list of 158 genes selected as biomarkers for the expression level at high frequency in the examples.

FIG. 13-3 shows a characteristic diagram demonstrating a list of 158 genes selected as biomarkers for the expression level at high frequency in the examples.

FIG. 13-4 shows a characteristic diagram demonstrating a list of 158 genes selected as biomarkers for the expression level at high frequency in the examples.

FIG. 13-5 shows a characteristic diagram demonstrating a list of 158 genes selected as biomarkers for the expression level at high frequency in the examples.

FIG. 13-6 shows a characteristic diagram demonstrating a list of 158 genes selected as biomarkers for the expression level at high frequency in the examples.

FIG. 13-7 shows a characteristic diagram demonstrating a list of 158 genes selected as biomarkers for the expression level at high frequency in the examples.

FIG. 13-8 shows a characteristic diagram demonstrating a list of 158 genes selected as biomarkers for the expression level at high frequency in the examples.

FIG. 13-9 shows a characteristic diagram demonstrating a list of 158 genes selected as biomarkers for the expression level at high frequency in the examples.

FIG. 14 shows a characteristic diagram demonstrating the correlation between the qLTG3-1 expression level and the fresh weight of the terrestrial part of the BIL line prepared in the examples and used for RNA-seq analysis and the parent variety.

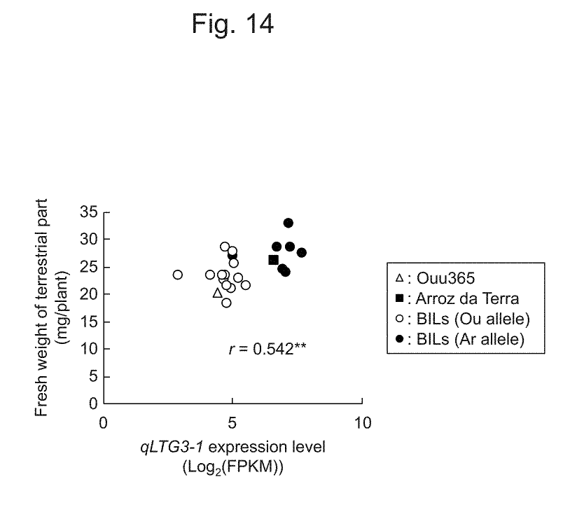

FIG. 15 shows a characteristic diagram demonstrating the correlation between the SG-1 expression level and the fresh weight of the terrestrial part of the BIL line prepared in the examples and used for RNA-seq analysis and the parent variety.

FIG. 16 shows a characteristic diagram demonstrating the correlation between the SG-1 expression level and the fresh weight of the terrestrial part of all the BIL104 lines prepared in the examples and the parent variety.

FIG. 17 shows a characteristic diagram demonstrating the correlation between the estimated fresh weight of the terrestrial part determined based on the expression level data and the fresh weight of the terrestrial part concerning the genes included in the list prepared in Example 1 and the measured fresh weight of the terrestrial part.

### DETAILED DESCRIPTION

Hereafter, the apparatus and/or the method for transcriptome analysis of the present disclosure are described in detail with reference to the drawings.

### The First Embodiment

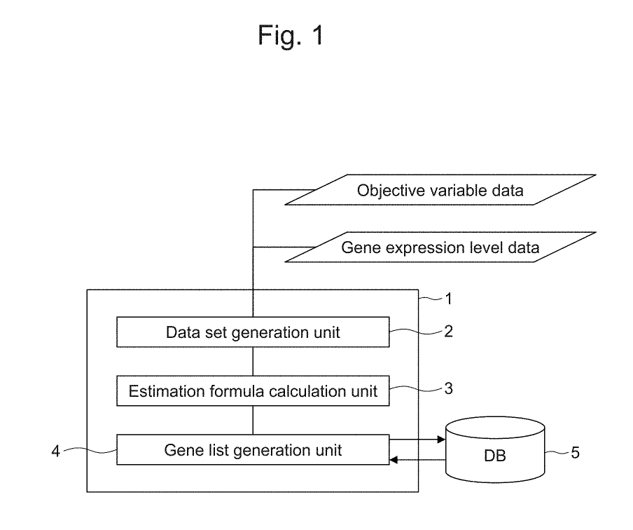

As shown in FIG. 1, the apparatus 1 for transcriptome analysis of the present disclosure comprises: the data set generation unit 2 that generates the first to the mth data sets (2≤m≤p−1) from data sets including a large quantity of gene expression level data (p-dimensional; p: the number of transcription products) for particular objective variable data; the estimation formula calculation unit 3 that applies the method of regularization to each of the first to the mth data sets generated in the data set generation unit 2 and calculates the first to the mth estimation formulae using the objective variable data as objective variables and the gene expression level data as explanatory variables; and the gene list generation unit 4 that generates a list of genes corresponding to the gene expression level data included in the first to the mth estimation formulae determined in the estimation formula calculation unit 3. The apparatus 1 for transcriptome analysis may be accessible to the external database 5 storing the annotation information of the genes.

The data set to be inputted into the apparatus 1 for transcriptome analysis includes given objective variable data and gene expression level data (p-dimensional data). The term “objective variable data” refers to numerical data concerning phenotypes including quantitative or qualitative traits, numerical data concerning the presence or absence of conditions of circumferential environment and an extent thereof, and numerical data concerning the presence or absence of the treatment provided for the target organism of analysis and an extent thereof More specific examples of objective variable data include data concerning the amount of target organisms of analysis, such as plants, produced (e.g., the weight of the terrestrial part, the weight of the root, the area of the leaf, and the seed yield) and data concerning stress imposed on target organisms of analysis (e.g., the duration of high-temperature treatment, the duration of low-temperature treatment, the concentration of drug treatment, and the duration of pest stress exposure).

The gene expression level data are numerical data indicating the relative expression level of the transcription product to be observed. Specific examples of gene expression level data include microarray data obtained with the use of commercially available microarrays for gene expression analysis (for transcriptome analysis) and the contract service for gene expression analysis provided in the market and sequence data obtained via expression analysis (RNA-Seq) using the next-generation sequence. In particular, the gene expression level data are preferably sequence data obtained via expression analysis (RNA-Seq) using the next-generation sequencer because the sequence data obtained via expression analysis (RNA-Seq) using the next-generation sequencer encompasses transcription products of target organisms.

With the use of the apparatus 1 for transcriptome analysis, a list of genes capable of describing the objective variable data can be generated by analyzing the data set described above. The list of genes is not limited to the list of genes encoding proteins in a narrow sense, and it encompasses a list of transcription products from the non-coding region.

When the early-stage growth level of a plant (the plant weight during a given period) is designated as the objective variable data, for example, a list of genes capable of describing the early-stage growth level can be generated with the use of the apparatus 1 for transcriptome analysis. When the concentration of a drug used to treat a plant is designated as the objective variable data, a list of genes expressed in accordance with the concentration of the drug can be generated with the use of the apparatus 1 for transcriptome analysis. When the temperature at the time of sampling is designated as the objective variable data, a list of genes expressed in accordance with the growth temperature can be generated with the use of the apparatus 1 for transcriptome analysis.

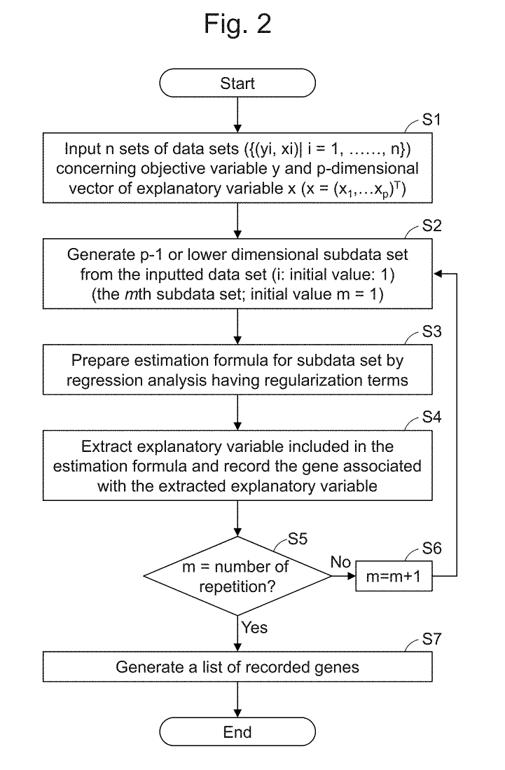

The apparatus 1 for transcriptome analysis of the constitution shown in FIG. 1 is capable of generating the list of genes in accordance with, for example, the flow chart shown in FIG. 2.

At the outset, the gene expression level data outputted by a microarray apparatus, next-generation sequencer, or the like and the objective variable data are inputted (Step S1). The inputted gene expression level data are designated as the p-dimensional vector of explanatory variables (x={x₁, . . . . . . , xₚ}). The inputted objective variable is designated as “y.” In this example, n sets of data sets ({(yᵢ, x₁)|i=1, . . . . . , n}) composed of the p-dimensional vector of explanatory variables x and the objective variable y are to be inputted.

In the data set generation unit 2, subsequently, the gene expression level data contained in the inputted n sets of data sets are sampled at random, so as to generate p−1 dimensional or lower subdata sets (Step S2). In this step, the mth subdata set designating the initial value as m=1 is generated. In other words, the generated mth subdata set is defined as a data set including a smaller number of gene expression level data than the inputted data sets as a result of random deletion of the gene expression level data contained in the inputted data set.

The generated mth subdata set may be a part of the gene expression level data (p-dimensional) included in the inputted data sets. For example, the mth subdata set may be 5% to 90%, 5% to 70%, 5% to 50%, 10% to 50%, 10% to 25%, or 10% to 15% of the p-dimensional gene expression level data.

When the number of gene expression level data is 30,000 (i.e., P=30,000: 30,000 transcription products), for example, the mth subdata set generated in the data set generation unit 2 can include randomly selected 1,000 to 20,000 gene expression level data, preferably 1,500 to 15,000 gene expression level data, more preferably 1,500 to 7,500 gene expression level data, and further preferably 1,500 to 4,500 gene expression level data.

In the estimation formula calculation unit 3, subsequently, the mth subdata set generated in the data set generation unit 2 is subjected to the method of regression analysis having regularization terms to calculate the mth estimation formula having the objective variable data as objective variables and the gene expression level data as explanatory variables (Step S3). The method of regression analysis having regularization terms is also referred to as the “regularization regression model,” which is a method of analysis comprising adding restrictions (penalties) to the least squares to shrink the estimator. Specific examples of methods of regression analysis having regularization terms include the method of LASSO regression analysis, the method of Ridge regression analysis, and the method of elastic net regression analysis. It is particularly preferable that the estimation formula be calculated in accordance with the method of LASSO regression analysis. When the method of LASSO regression analysis is adopted, in particular, the parameter of the gene expression level data that are not important to describe objective variables is designated as 0 in the estimation formula calculated in this step.

When calculating an estimation formula in accordance with the method of LASSO regression analysis, a reference may made to Friedman et al., Regularization Paths for Generalized Linear Models via Coordinate Descent, Journal of Statistical Software, January 2010, Volume 33. Issue I.

In the gene list generation unit 4, subsequently, the gene expression level data included in the estimation formula calculated in the estimation formula calculation unit 3 are extracted and genes corresponding to the extracted gene expression level data are recorded (Step S4). Since the estimation formula is calculated by the method of regression analysis having regularization terms, specifically, the gene expression level data that are important to describe objective variables can be selectively extracted. When the method of LASSO regression analysis is adopted as the method of regression analysis having regularization terms, for example, gene expression level data other than the gene expression level data with the parameter designated as 0 are extracted.

In Step S5, subsequently, whether or not the processing of Steps S2 to S4 were repeated the predetermined number of times (in times) is determined. When the number of repetition was determined as 10,000 in advance (m=10,000), for example, the first subdata set with the initial value designated as 1 was subjected to the processing of Steps S2 to S4. In Step S5, subsequently, the value (m=1) is compared with 10,000 and the processing proceeds to Step S6. In Step S6, the in value is increased by 1, and Steps S2 to S5 are repeated until the m value reaches 10,000.

By repeating the Steps S2 to S6 the in number of times, the 1st to the mth subdata sets of the n sets of data sets inputted in Step S1 can be appropriately subjected to the first to the mth estimation formulae.

In Step S7, subsequently, a list of genes recorded in Step S4 with respect to the first to the mth estimation formulae is outputted in the gene list generation unit 4. The list of genes generated in the gene list generation unit 4 is not particularly limited, genes corresponding to the gene expression level data extracted in the gene list generation unit 4 may be enumerated, or the list may have format comprising the correlation between the genes corresponding to the extracted gene expression level data and the appearance probability of the gene. The appearance probability can be calculated as the frequency of a particular gene to be included relative to the number of all the genes included in the first to the mth estimation formulae.

The list of genes generated in the gene list generation unit 4 may selectively include genes exhibiting the appearance probability exceeding a given level. Alternatively, the list may include genes in descending order of the appearance probability.

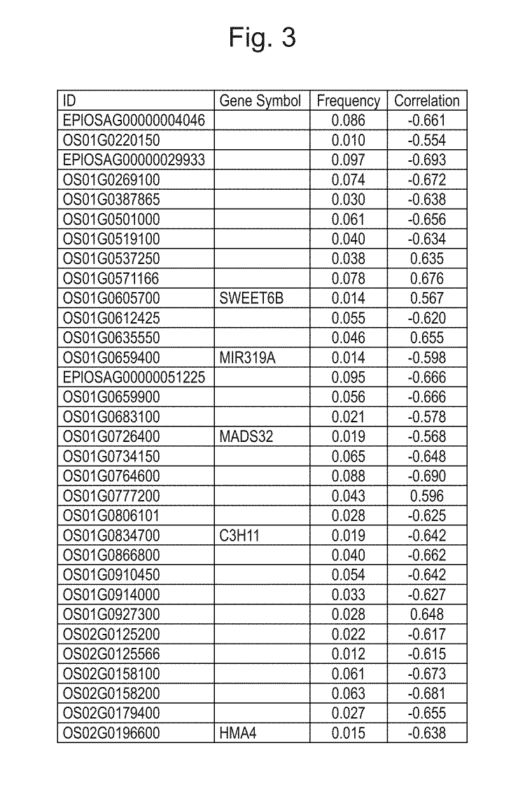

FIG. 3 shows an output example of the list of genes generated in the gene list generation unit 4. As shown in FIG. 3, the list of genes generated in the gene list generation unit 4 includes IDs assigned to the transcription products, symbols indicating the genes from which the transcription products are derived, the appearance probability calculated for each transcription product (Frequency) and the correlation coefficient with objective variable data.

As shown in FIG. 1, in addition, the apparatus 1 for transcriptome analysis may access the external database 5 to search for the annotation information of the genes included in the list and the list may have format comprising the obtained annotation information in connection with the genes of interest. Alternatively, the apparatus 1 for transcriptome analysis may classify the genes into groups based on the obtained annotation information and generate a list of each group of genes.

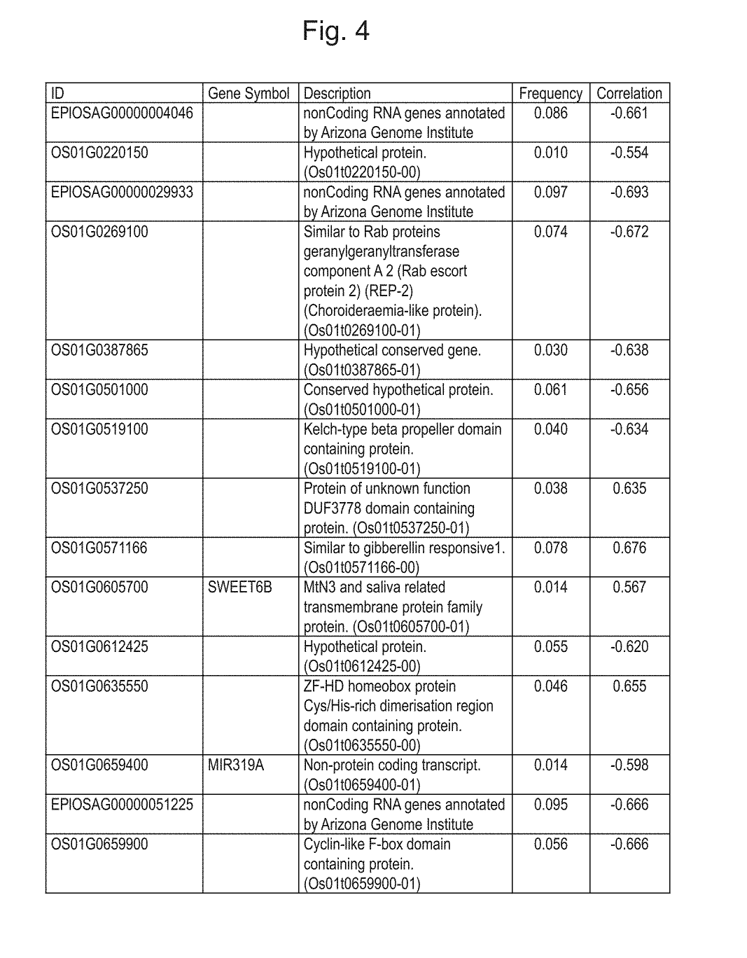

FIG. 4 shows an output example of the list of genes generated in the gene list generation unit 4. As shown in FIG. 4, the list of genes generated in the gene list generation unit 4 includes IDs assigned to the transcription products, symbols indicating the genes from which the transcription products are derived, information concerning functions of the genes identified by the symbols, the appearance probability calculated for each transcription product (Frequency) and the correlation coefficient with objective variable data.

According to the lists of the genes shown in FIG. 3 and/or FIG. 4, the genes that can describe the objective variable data can be understood as a result of analysis of particular objective variable data. When the appearance probability is correlated with the lists of the genes, in particular, a degree of the correlation between each of the listed genes and the objective variable data can be evaluated on the basis of the appearance probability. When the annotation information is correlated with the lists of the genes, in addition, the correlation between each of the listed genes and the objective variable data can be understood in a biological sense on the basis of the annotation information.

### The Second Embodiment

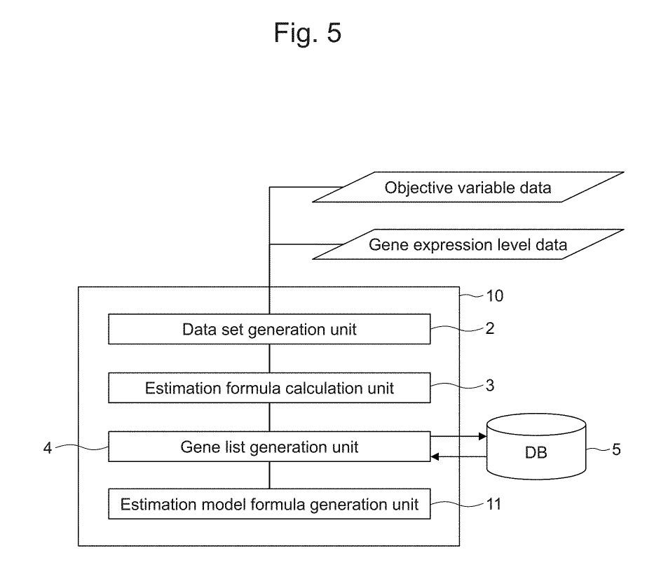

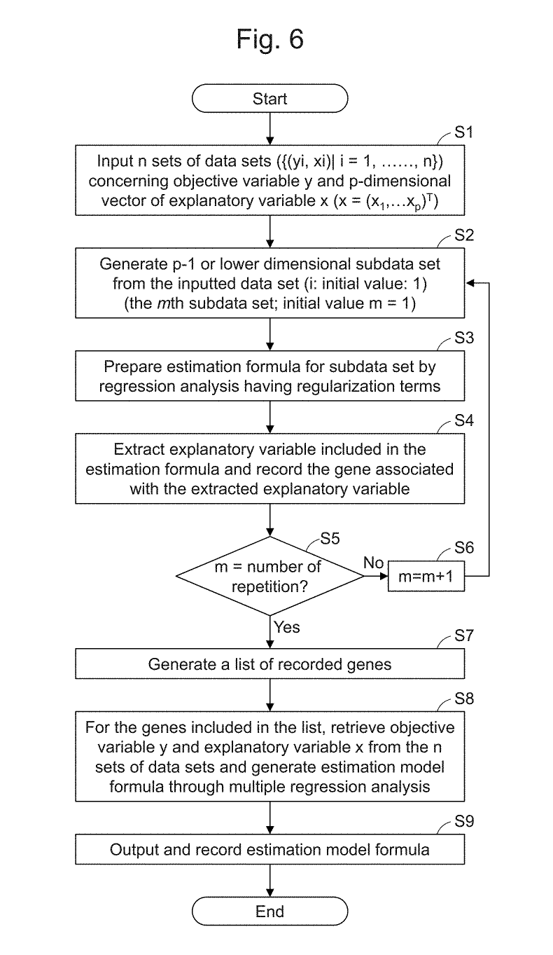

The apparatus and the method for transcriptome analysis of the present disclosure are not limited to those according to the first embodiment described above. As shown in FIGS. 5 and 6, the apparatus and the method for transcriptome analysis of the present disclosure can use the list of genes prepared concerning particular objective variable data to prepare an estimation model formula concerning the objective variable data. Concerning the apparatus 10 for transcriptome analysis and the method of analysis shown in FIGS. 5 and 6, descriptions concerning the constitution and the steps identical to those concerning the apparatus and the method for transcriptome analysis shown in FIGS. 1 and 2 are omitted herein by using the same references used in FIGS. 1 and 2.

The apparatus 10 for transcriptome analysis shown in FIG. 5 comprises the estimation model formula generation unit 11 that generates an estimation model formula based on the genes included in the list generated in the gene list generation unit 4. An apparatus for transcriptome analysis comprising the estimation model formula generation unit 11 is capable of generating an estimation model formula including explanatory variables describing given objective variable data, in addition to the list of genes generated in the gene list generation unit 4 (e.g., FIGS. 3 and 4).

As shown in FIG. 6, the apparatus 10 for transcriptome analysis generates lists of genes in Step S1 to S6 as with the case of the first embodiment described above. Thereafter, the apparatus 10 for transcriptome analysis retrieves explanatory variable data and objective variable data concerning the genes included in the list from the n sets of data sets inputted in Step S1 in the estimation model formula generation unit 11. As a result of multiple regression analysis or machine learning with the use of the objective variables y and the explanatory variables x concerning the relevant genes, estimation model formulae describing give objective variable data can be constructed.

In the estimation model formula generation unit 11, an estimation model formula describing given objective variable data may be generated through multiple regression analysis or machine learning with the use of objective variables y and explanatory variables x concerning all the genes included in the list generated in the gene list generation unit 4. Alternatively, an estimation model formula describing given objective variable data may be generated through multiple regression analysis or machine learning with the use of objective variables y and explanatory variables x concerning a part of the genes included in the list. Examples of a part of genes included in the list include genes exhibiting the expression frequency over the threshold or genes correlated with given annotation information.

A method for constructing an estimation formula is not particularly limited, and examples include multiple regression analysis, such as the method of LASSO regression analysis, the method of Ridge regression analysis, and the method of elastic net analysis, and machine learning, such as Random forest analysis and deep learning.

For example, an estimation model formula can be prepared by the Random forest method in the estimation model formula generation unit 11. The estimation model formula prepared by the Random forest method is a model formula in the form of a decision tree that calculates a given objective variable y and is prepared as a function for the expression level data x of the gene included in the list generated in the gene list generation unit 4. With the estimation model formula generated in the estimation model formula generation unit 11, the estimated values for the objective variables concerning a given organism can be determined on the basis of the gene expression level data obtained for the organism.

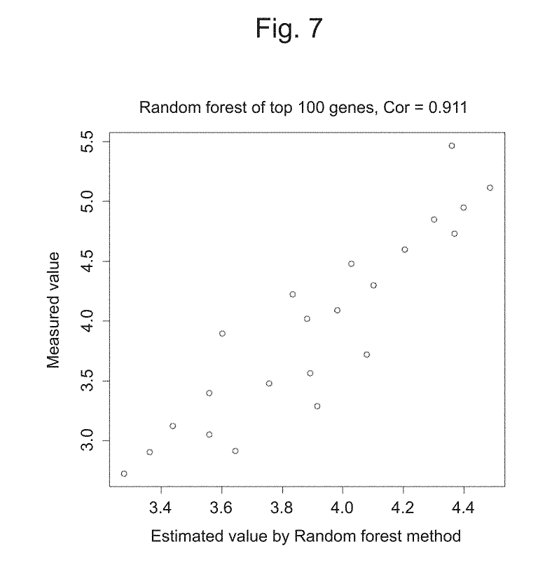

FIG. 7 shows the correlation between the objective variables y (measured values) included in the list generated in the gene list generation unit 4 and the estimated values calculated using the estimation model formula generated by the Random forest method. As shown in FIG. 7, the estimated values are found to be highly compatible with the measured values according to the estimation model formula prepared by adopting the Random forest method. The chart shown in FIG. 7 indicates the use of the data described in the examples below.

When the estimation model formula is constructed by designating the plant's seed yield as an objective variable, for example, the plant's seed yield can be estimated with the use of the gene expression level data obtained from the target plant. Specifically, the objective variable, such as the seed yield, can be estimated based on the gene expression level data that are readily obtained using a next-generation sequencer without performing cultivation tests of the target plant.

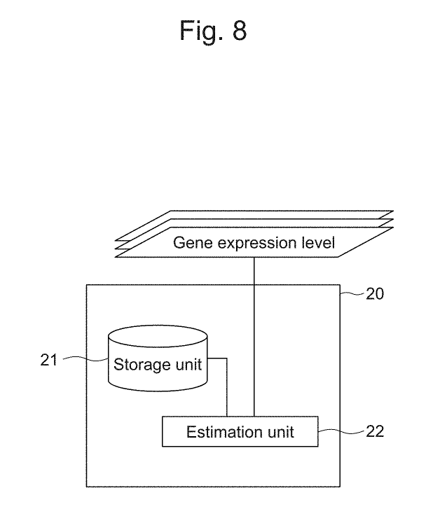

With the use of the apparatus for transcriptome analysis according to the present embodiment, as described above, an estimation model formula concerning a given objective variable can be prepared. With the use of the estimation model formula prepared, for example, the property evaluation system 20 for the target organism can be constructed as shown in FIG. 8.

The property evaluation system shown in FIG. 8 comprises: the storage unit 21 that stores the estimation model formula concerning a given objective variable prepared with the use of the apparatus for transcriptome analysis according to the present embodiment; and an estimation unit 22 that estimates the objective variable on the basis of the gene expression data concerning the target organism. The storage unit 21 stores estimation model formulae for various objective variables of each target organism. For example, the storage unit 21 can store estimation model formulae for objective variables, such as the weight of the terrestrial part, the weight of the root, the leaf area, the seed yield, the duration of high-temperature treatment, the duration of low-temperature treatment, the concentration of drug treatment, and the duration of exposure to disease and pest stress, of the target plant.

When the gene expression level data concerning the target organism are inputted, the estimation unit 22 assigns the gene expression level data into the relevant estimation model formula stored in the storage unit 21, and it can determine the estimated values for various objective variables.

As described above, the property evaluation system 20 stores estimation model formulae for various objective variables in the storage unit 21. On the basis of the gene expression level data of the target organism, accordingly, the estimated values concerning the objective variables can be outputted. When the gene expression level data concerning a given plant are inputted, for example, estimated values concerning the objective variables, such as the weight of the terrestrial part, the weight of the root, the leaf area, the seed yield, the duration of high-temperature treatment, the duration of low-temperature treatment, the concentration of drug treatment, and the duration of exposure to disease and pest stress, can be collectively or selectively obtained.

The apparatuses and the methods for transcriptome analysis according to the first embodiment and the second embodiment described above can be realized by, for example, a computer comprising a central processing unit (CPU), a main storage unit, an auxiliary storage unit, an output unit, and an input unit. For example, objective variable data and gene expression level data can be inputted through the input unit under the control of CPU and stored in the main storage unit or in the auxiliary storage unit. For example, the mth subdata set can be generated under the control of CPU in accordance with a given algorithm. In addition, the mth estimation formula based on the mth subdata set can be generated under the control of CPU in accordance with a given algorithm. Thus, the apparatuses and the methods for transcriptome analysis according to the first embodiment and the second embodiment described above can be realized under the control of CPU.

The apparatuses and the methods for transcriptome analysis according to the first embodiment and the second embodiment described above can be realized by so-called cloud computing. In cloud computing, for example, objective variable data and gene expression level data stored in a cloud server can be used, and the generated estimation formulae and gene lists can be stored in the cloud server.

### EXAMPLES

Hereafter, the present disclosure is described in greater detail with reference to the following examples, although the technical scope of the present disclosure is not limited to these examples.

### Example 1

### 1. Material and Method

### 1-1. Rice Line as Experimental Material and Cultivation Condition

In this example, Ouu 365/Arroz da Terral/Guu 365 backcross inbred lines (Bits) described in Fukuda et al., 2014, Plant Production Science 17: 41-46 were used. Seeds of the lines were disinfected with a 50-fold dilution of hypochlorite, washed 3 times with tap water, and then soaked in water at 30° C. for 2 days for germination. Germinated seeds (24 seeds per line) were sowed in a hydroponic float system (Fukuda et al., 2012, Plant Production Science 15: 183-191) and allowed to grow in a hydroponic solution (Hayashi and Chino, 1986, Plant and Cell Physiology 27: 1387-1393). The hydroponic solution was replaced with a fresh solution every 2 days, and the plants were allowed to grow at 20° C. in a growth chamber in a light-dark cycle of 12 hours for 14 days.

### 1-2. Method

### 1-2-1. QTL Analysis

Seedlings of the BIL104 lines and 2 parent lines were sampled 14 days after germination and dehydrated in a dryer at 80° C. for 2 days, Thereafter, seeds and roots were removed and the resulting plants were weighed. An experiment was composed of 3 biological replicates, and the average dry weight of the terrestrial parts of the seedlings was used for QTL analysis. BIL genotypes were analyzed with the use of 124 types of SSR markers (Fukuda et al., 2014, Plant Production Science 17: 41-46), and QTL analysis was carried out with the use of MAPMAKER/EXP 3.0 (Lander et al., 1987, Genomics 1: 174-181, doi: 10.1016/0888-7543 (87) 90010-3) and QTL Cartographer 2.5 (Wang et al., 2010, Statistical Genetics & Bioinformatics, provided by North Carolina State University).

### 1-2-2. RNA Isolation and RNA-seq

Parent lines (Ouu365 and Arroz da Terra) and BIL20 lines with different early-stage grow levels were selected and subjected to RNA-seq analysis. Seeds and roots were removed from the seedlings 14 days after germination, the fresh weight of the terrestrial parts of the seedlings was measured, and the seedlings were frozen in liquid nitrogen, followed by storage at −80° C. before analysis. RNA was extracted using the RNeasy mini Kit (manufactured by Qiagen) and RNA-seq analysis was then performed. After quantitative and qualitative RNA analyses were carried out with the use of the 2100-Bioanalyzer (manufactured by Agilent Technologies), a sequencing library was prepared with the use of the TruSeq RNA LT Sample Preparation Kit v2 (manufactured by Illumina Inc.). The library was subjected to 100-bp single-end read cycles of sequencing on Illumina Hiseq 2000. Fastq files of the sequencing results are shown in DDBJ Sequence Read Archive (DRA), Accession No. DRA006312.

The sequence data were subjected to mapping with the use of TopHat2 (Kim et al., 2013, Genome Biology 14: 13, doi: 10.1186/gb-20 13-14-4-r36; Trapnell et al., 2009, Bioinformatics 25: 1105-1111. doi:10.1093/bioinformatics/btp120) using the Oryza saliva-Nipponbare-Reference-IRGSP-1.0 genome (Oryza saliva.IRGSP-1.0.24.dna.toplevel.fa.gz, ftp://ftp.ensemblgenomes.org/pub/release-24/plants/fasta/oryza_sativa/dna/) and the gene set (Oryza sativa, IRGSP-1.0.24.gtf.gz, ftp://ftp.ensemblgenomes.org/pub/release-24/plants/gtf/oryza_sativa/) as the reference sequences. Each gene expression level was calculated in terms of the fragments per kilobase million (FPKM) value.

### 1-2-3. Biomarker for Expression Level and Calculation of Appearance Probability of Gene

The biomarker for the expression level representing the fresh weight of the terrestrial part of the seedling and the appearance probability of gene were examined in the manner described below. The genes (37,043 species) exhibiting the average expression level of 0.01 or higher were subjected to the analysis in the manner described below. Each gene expression level was converted into the Log 2 value after 0.01 was added to the FPKM value. The biomarker for the expression level was selected using the L1-formed regression model by the LASSO method (Tibshirani, 1996, Journal of the Royal Statistical Society Series, B-Methodological 58: 267-288). In order to calculate the biomarker gene appearance probability, biomarker selection was repeated with the use of a transcriptome subset. From among 37,043 genes, 10% thereof were randomly selected and used for LASSO analysis as variables. From among the inputted variables, 8 genes were selected as appropriate explanatory variables exhibiting a coefficient other than zero and designated as the biomarkers for the expression level. Selection of the subset and quantification of the biomarkers for the expression levels were repeated 10,000 times. The frequency for each gene to be selected as a biomarker in 10,000 trials was determined. Analysis was carried out using the R glmnet package (R. Core Team, 2015, R: A language and environment for statistical computing, https://www.R-project.org/).

### 1-2-4. SG1 Gene Sequencing

The SG1 gene coding regions of Ouu365 and of Arroz da Terra and upstream 2,108-bp regions were amplified via PCR using the primers: 5′-GGGACGTGATAACCGACTCA-3′ (SEQ ID NO: 1) and 5′CCCCACTGTACGTTCTCTCC-3′ (SEQ ID NO: 2). The PCR products were purified using the illustra ExoProStar kit and sent to Fasmac Co., Ltd. for sequencing.

In order to detect single nucleotide substitution at a site 1,948-bp upstream from the translation initiation site, the PCR product was amplified using the following primers: 5′-GGGACGTGATAACCGACTCA-3′ (SEQ ID NO: 3) and 5′- TTCAGGTCACCTAGCCCATC-3′ (SEQ ID NO: 4) and cleaved with the restriction enzyme HaeIII. While the Arroz da Terra sequence (GGCC) was cleaved with HaeIII, the Ouu365 sequence (AGCC) was not cleaved.

### 1-2-5. Quantitative Real-Time PCR

Total RNA was extracted from the terrestrial part of the seedling in the manner described above. cDNA was synthesized. from 1 μg of total RNA using the PrimeScript RT reagent Kit with gDNA Eraser (Takara Bio). The SG1 cDNA level was quantified via real-time PCR using the Thermal Cycler Dice Real Time System III, SYBR Premix Ex Taq, and the primer set OA045647 (Takara Bio). Real-time PCR was composed of 3 technical replicates. In order to determine the copy number of SG1 mRNA, the PCR product of SG1 was amplified using cDNA of Ouu365 as a template and the following primers: 5′-CGACCAGCTGATCTCCAA-G3′ (SEQ ID NO: 5) and 5-CATTTTTACTGGCCCTTCCA-3′ (SEQ ID NO: 6), and the amplified product was used as a standard for real-time quantitative PCR. The standard PCR product was quantified using the Qubit fluorometer (Thermo Fisher Scientific), and the copy number was determined based on the molecular weight. The SG1 expression level (copies per ng RNA) was converted into the Log 2 value and then used for QTL analysis.

### 2. Results

### 2-1. QTL Analysis of Backcross Inbred Line (BIL)

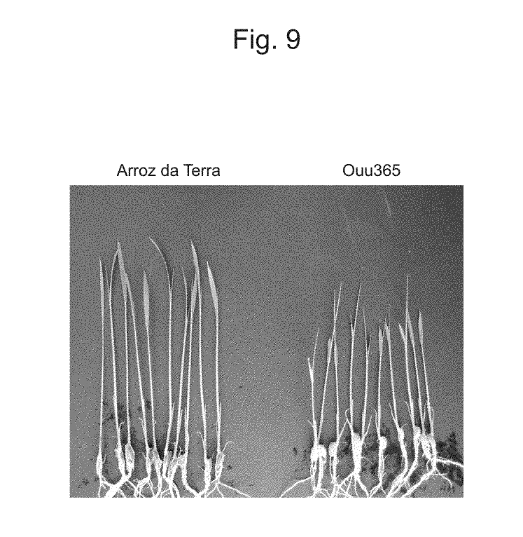

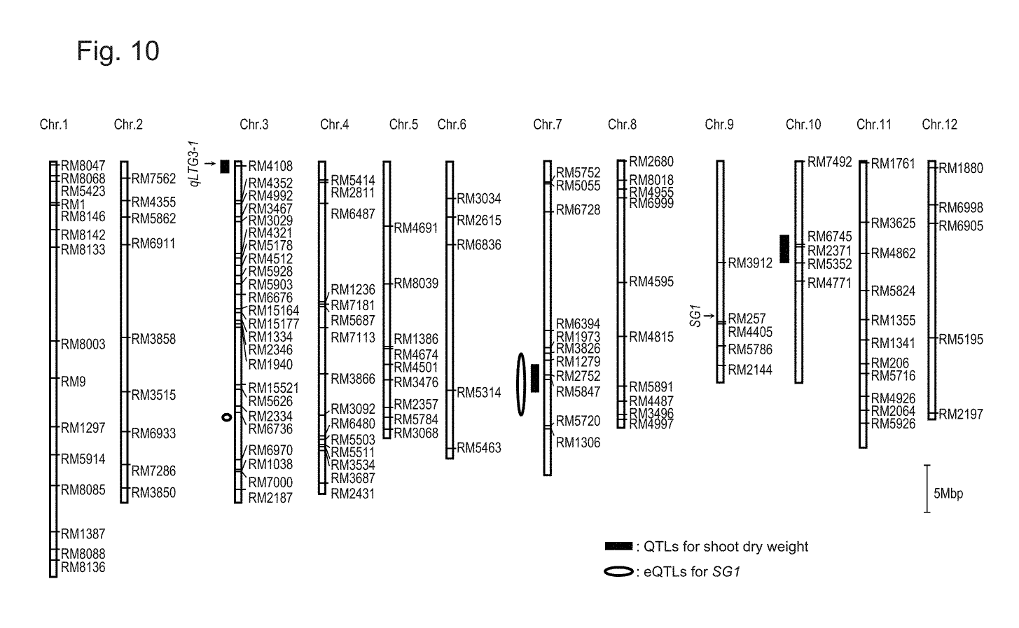

The average dry weight of the terrestrial part of Arroz da Terra and that of Ouu365 were 5.11 mg and 2.91mg, respectively, 14 days after germination. That is Arroz da Terra was significantly heavy (t-test; significance: 5%). The dry weight of the terrestrial part of the BIL104 line was between 2.52 mg and 5.47 mg (FIG. 9). The dry weight of the terrestrial part of the BIL104 line was subjected to QTL analysis. As a result, QTL that would increase the dry weight of the terrestrial part was detected in the chromosomes 3, 7, and 10 of Arroz da Terra (Table 1, FIG. 10). In FIG. 10, a black square indicates a position of QTL that increases the dry weight of the terrestrial part. A hollow oval indicates a position of eQTL that lowers the SG1 expression level.

TABLE 1

| | | Adjacent | | | |
|---|---|---|---|---|---|
| Traits | Chromosome | marker | LOD | Effects | R² (%) |
| Dry weight of | 3 | RM4108 | 3.10 | 0.20 | 8.2 |
| terrestrial part | 7 | RM2752 | 4.39 | 0.22 | 12.4 |
| | 10 | RM2371 | 3.17 | 0.20 | 9.0 |

### 2-2. RNA-seq Analysis and Selection of Biomarker Gene

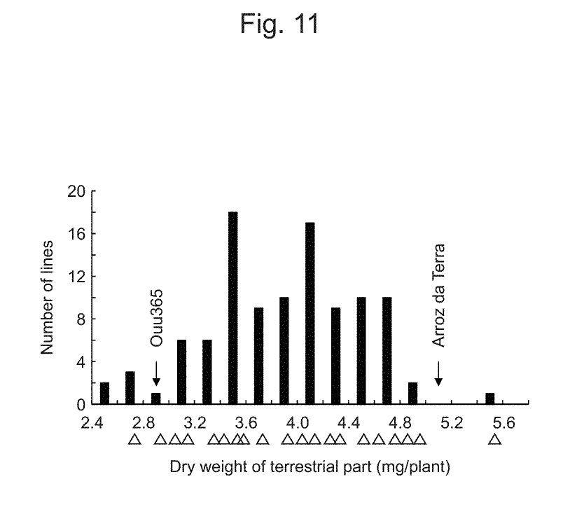

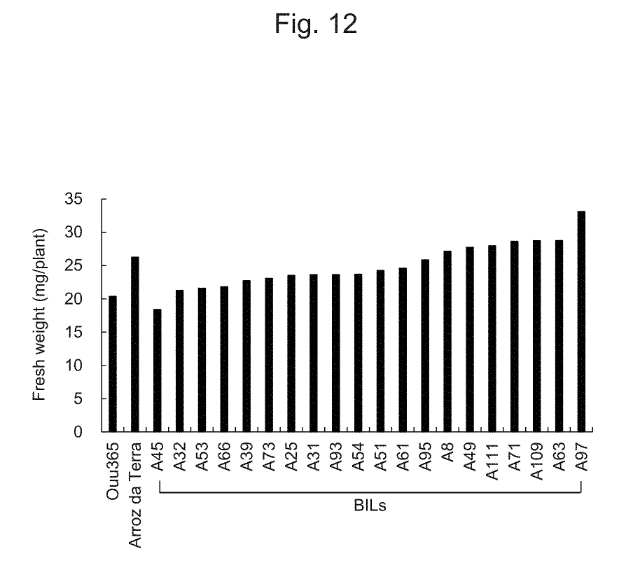

In order to search for transcription products correlated with the early-stage growth level, 2 parent lines and 20 BILs having different early-stage growth levels were used (FIG. 11), RNAs were extracted from the terrestrial parts of the seedlings 14 days after germination, and the extracted RNAs were then subjected to RNA-seq analysis. In FIG. 11, a hollow triangle indicates the average dry weight of the terrestrial part of each BIL line used for seq analysis. FIG. 12 shows the fresh weight of the terrestrial part of the seedling.

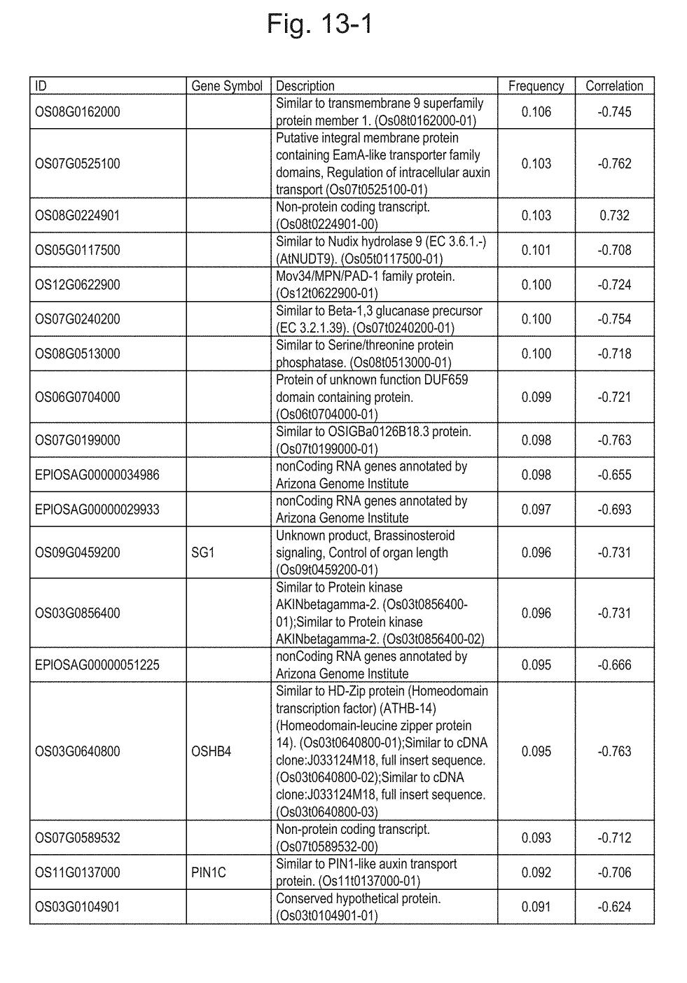

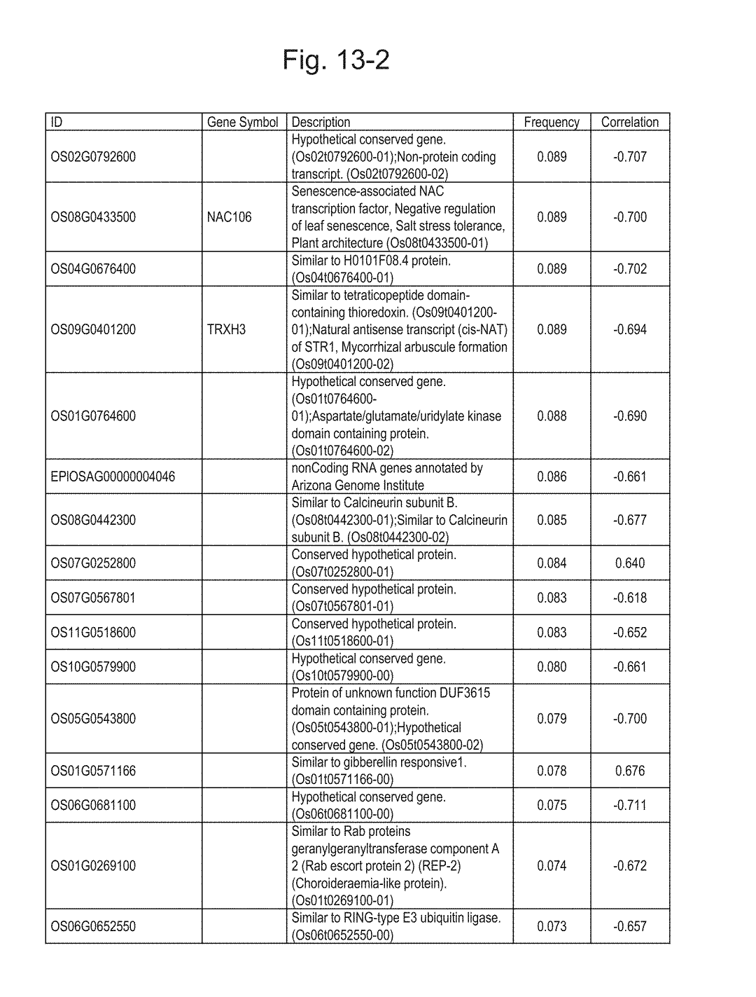

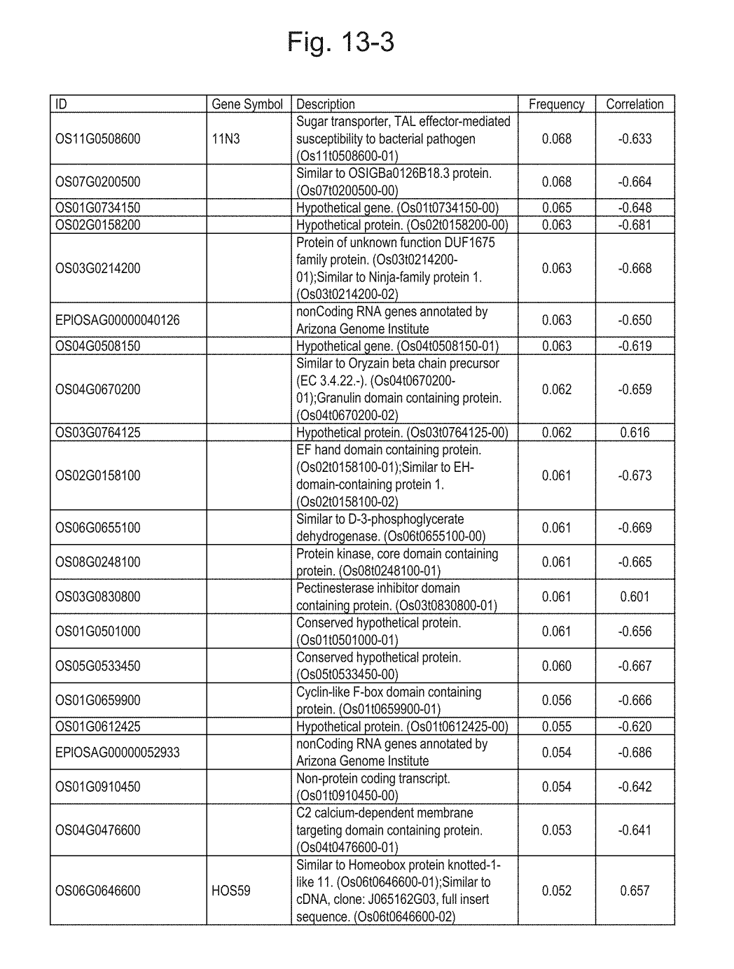

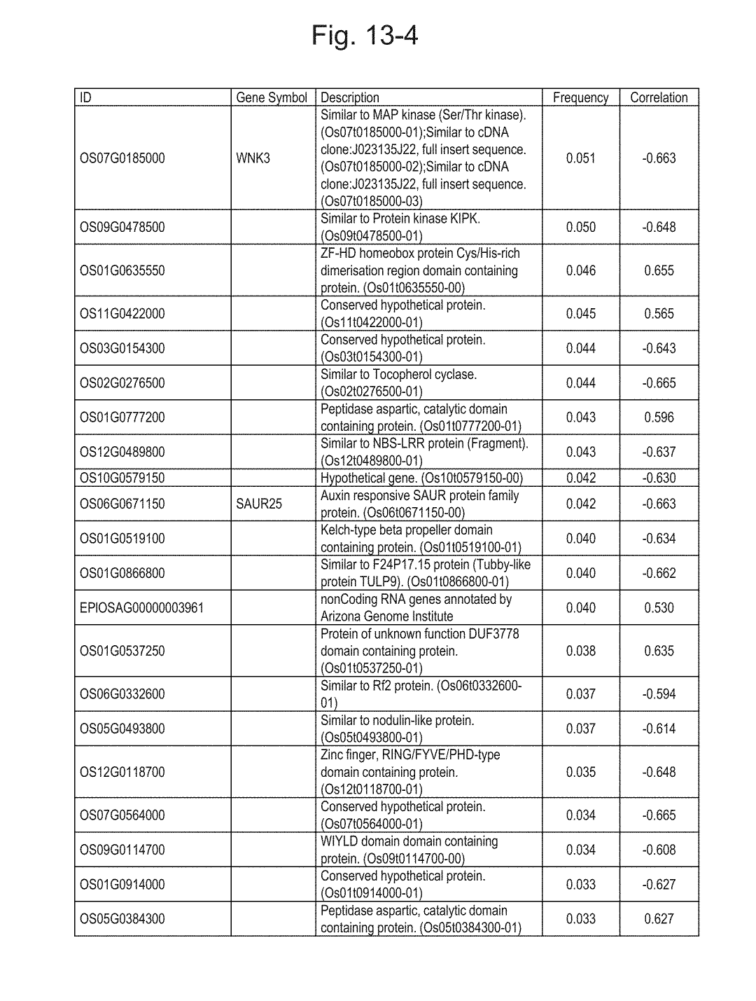

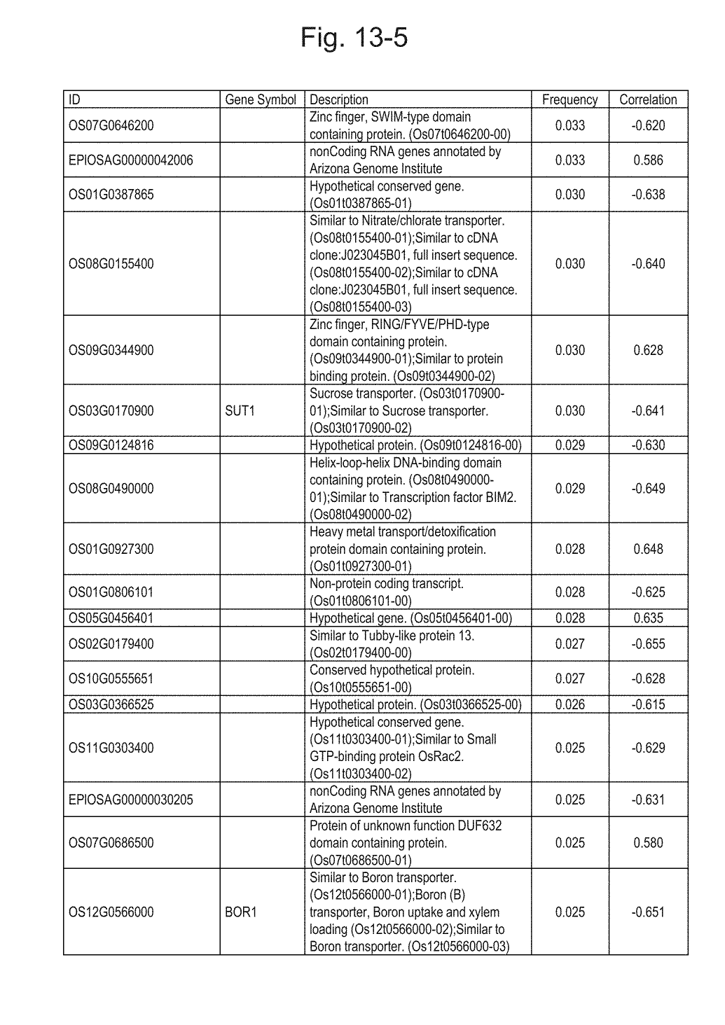

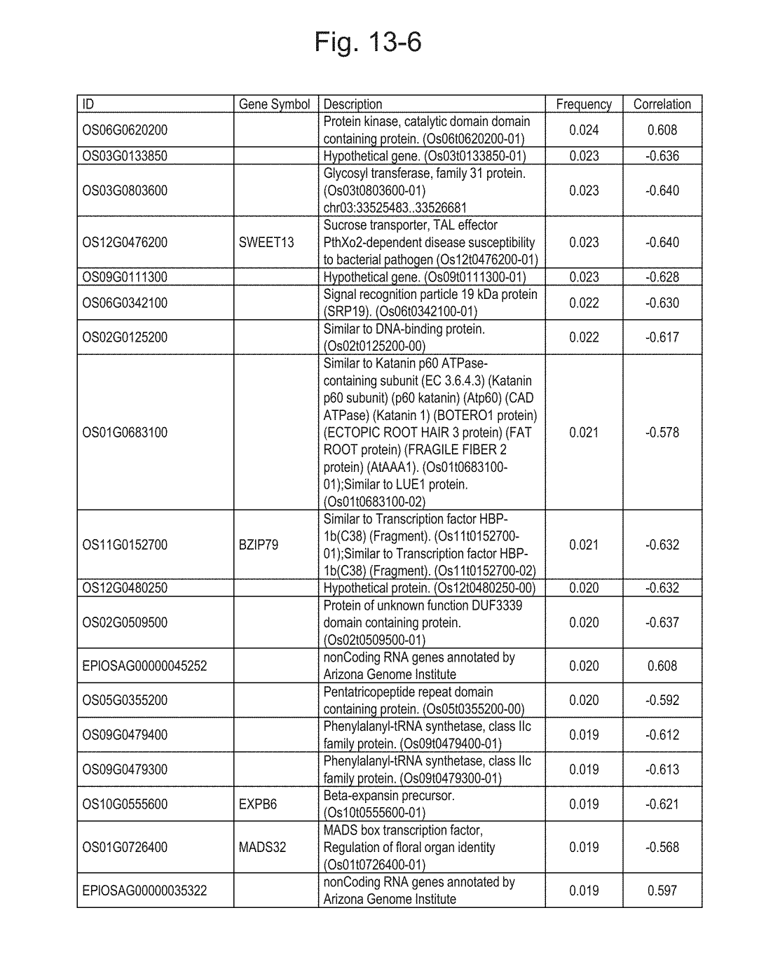

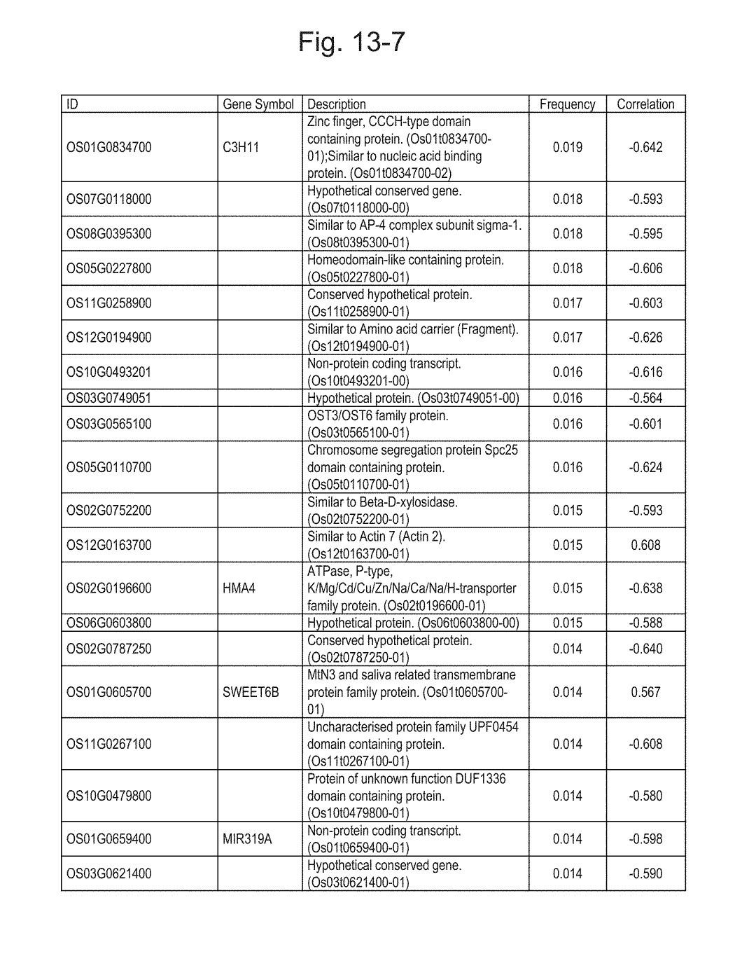

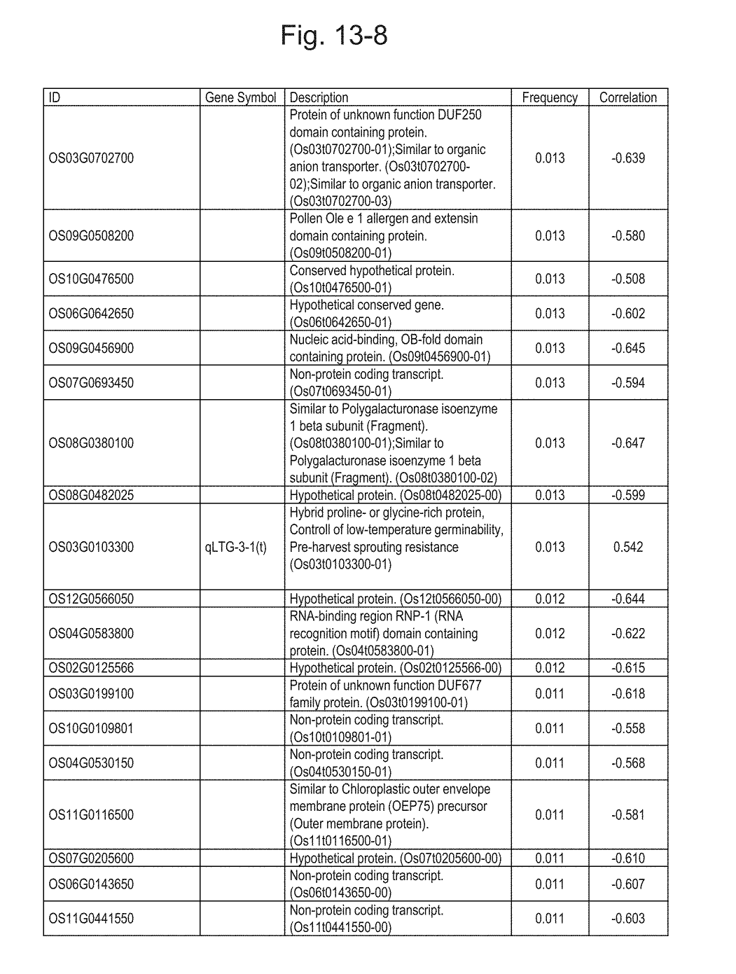

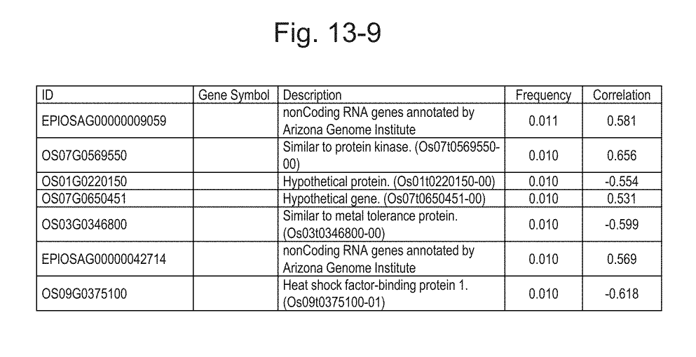

The number of reads was 41.6 M on average per sample, and 40.0 M reads/sample accounting for 96.1% of the total reads were mapped on the Os-Nipponbare-Reference-IRGSP-1.0 genome. The gene expression level was determined in terms of FPKM (fragments per kilobase of coding sequence per million reads). The appearance probabilities for genes serving as biomarkers indicating the fresh weights of the terrestrial parts of the seedlings was determined in the manner described below by the method described in “1-2-3. Biomarker for expression level and calculation of appearance probability of gene” above. From among all the expressed genes, 10% of the genes was randomly selected to constitute a subset, 8 genes were selected from the subset via LASSO analysis as explanatory variables indicating the fresh weights of the terrestrial parts of the seedlings (biomarkers for the expression levels). Selection of the subset and quantification of the biomarkers for the expression levels were repeated 10,000 times, and the frequency for each gene to be selected as a biomarker for the expression level was determined. A gene selected at high frequency indicates that the expression level thereof is correlated with the fresh weight of the terrestrial part of the seedling. At high frequency, 158 genes were selected (with a probability of 1% or higher). FIG. 13 shows a list of these selected 158 genes. The expression levels of the selected 158 genes were significantly correlated with the fresh weight of the terrestrial part (significance: 5%).

2-3. Biomarker Genes Selected at High Frequency Located Inside QTL Associated with the Weight of the Terrestrial Part of the Seedling

The genes selected at high frequency and the QTL associated with the weight of the terrestrial part of the seedling were examined, and the number of the genes located inside QTL of the chromosomes 3, 7, and 10 was 5, 6, and 4, respectively. The known gene associated with low-temperature germination (gLTG3-1) was included in the gene in the chromosome 3 (RAP ID: Os03g0103300, Fujino et al., 2008, Theoretical and Applied Genetics 108: 794-799, doi: 10.1007/s00122-003-1509-4). A significant positive correlation was observed between the qLTG3-1 expression level and the fresh weight of the terrestrial part of the lines used for RNA-seq 14). While a parent line (Arroz da Terra) is reported to have a functional gLTG3-1 gene (Fujino and Iwata, 2011, Theoretical and Applied Genetics 123: 1089-1097, doi: 10.1007/s00122-011-1650-4), another parent line (Ouu365) is found to lack a 71-bp site in the qLTG3-1 gene coding region and lack functions thereof (Fukuda et al., 2014, Plant Production Science 17: 41-46). The qLTG3-1 genotype of the BIL line used for RNA-seq was examined. As a result, the fresh weight of the terrestrial part and the qLTG3-1 expression level of the line having Arroz da Terra-type qLTG3-1 were found to be significantly higher than those of the line having the Ouu365-type qLTG3-1(t-test: significance: 1%).

2-4. Biomarker Genes Selected at High Frequency Located Outside QTL Associated with the Weight of the Terrestrial Part of the Seedling

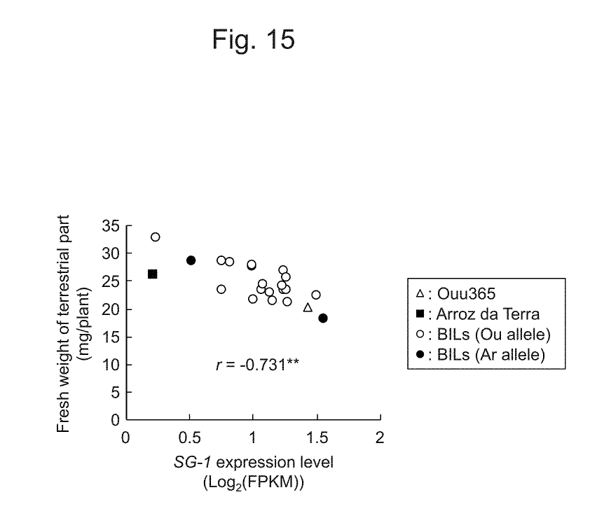

Among the genes selected at high frequency that were not located inside the QTL associated with the weight of the terrestrial part of the seedling, the known gene associated with inhibition of tissue elongation, SGI (Short Grain 1, RAP ID: Os09g045920 0, Nakagawa et al., 2012, Plant Physiology 158: 1208-1219, doi: 10.1104/pp. 111.187567), was included. A significant negative correlation was observed between the SG1 gene expression level and the fresh weight of the terrestrial part of the lines used for RNA-seq (FIG. 15). SGI is known to lower reactivity of a plant hormone to brassinosteroid and retard the plant growth in SG1-overexpressing transformants (Nakagawa et al., 2012, Plant Physiology 158: 1208-1219, doi:10.1104/pp. 111.187567). However, it has not been reported whether or not SGI has a natural mutation. As a result of comparison of nucleotide sequences of the SGI genes of the parent lines Arroz da Terra and Ouu365, no mutation, such as nucleotide substitution, deletion, or insertion, was observed in the coding region. While single nucleotide substitutions were observed at sites 1,948-b and 2,038-b upstream from the translation initiation point, the SG1 gene expression level of the line used for RNA-seq analysis was not influenced by the genotype at this position.

### 2-5. Quantitative Real-Time PCR Analysis of SGI Expression Level of BIL104 Line

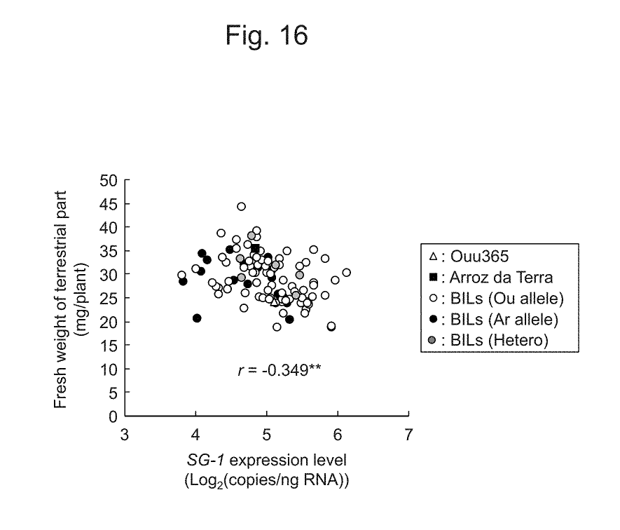

All the BIL104 lines and parent lines were subjected to quantitative real-time PCR to measure the SG1 expression levels in order to examine whether or not BIL lines other than those subjected to RNA-seq analysis would show the correlation between the SGI expression level and the weight of the terrestrial part of the seedlings. As a result, a significant negative correlation was observed between the SGI expression level and the fresh weight of the terrestrial part of the BIL104 lines and the parent lines (FIG. 16). No differences were observed in SGI expression level caused by the genotype at a site 1,948-b upstream of the translation initiation point. In order to inspect the chromosomal region affecting the SG1 expression level, the SG1 expression level of the BIL104 line was subjected to QTL analysis (eQTL analysis). As a result, eQTL that would lower the SGI expression level was detected at 2 sites (i.e., chromosome 3 and chromosome 7) of the Arroz da Terra line (Table 2 and FIG. 10). eQTL on chromosome 7 with strong activity was found to be located at the same position as QTL associated with the weight of the seedling (FIG. 10). No eQTL was detected on chromosome 9 in which the SG1 was present.

TABLE 2

| | | Adjacent | | | |
|---|---|---|---|---|---|
| Traits | Chromosome | marker | LOD | Effects | R² (%) |
| SG1 | 3 | RM6736 | 2.90 | −0.20 | 9.6 |
| expression | 7 | RM2752 | 6.13 | −0.23 | 18.4 |

This example demonstrates that candidate biomarker genes serving as indicators for the early-stage growth can be selected via RNA-seq analysis using the BIL20 lines and the parent lines. In addition, the known gene associated with inhibition of tissue elongation (SG1) was found to be included therein. While SGI is found to have effects of inhibiting tissue elongation in SG1-overexpressing transformants by activation tagging (Nakagawa et al., 2012, Plant Physiology 158: 1208-1219, doi: 10.1104/pp. 111.187567), whether or not SG1 expression levels vary between lines in a natural state remains unknown. The transcriptome analysis performed in this example demonstrates that there is a negative correlation between the SG1 expression level and the fresh weight of the terrestrial part of the seedling of the BIL lines (FIG. 15). In addition to the lines used for RNA-seq, also, negative correlations were observed between the SG1 expression level and the fresh weight of the terrestrial part of the seedling in all the 104 BIL lines as a result of quantitative real-time PCR analysis (FIG. 16). This indicates that SG1 affects the early-stage growth level of a seedling. On the basis of the results provided above, the transcriptome analysis of this example involving the use of RNA-seq data of the 22 lines is considered to be an effective means for detecting transcription products associated with the early-stage growth.

2-6. Usefulness of transcriptome analysis demonstrated in this example

While an extensive analysis of transcription products (i.e., transcriptome analysis) is a powerful tool capable of detecting transcription products that would affect various morphological and physiological traits, transcription products are influenced by many environmental and genetic factors in complicated ways. In order to statistically select biomarkers for expression levels indicating particular traits, accordingly, it is considered preferable that a large number of samples (e.g., hundreds or more) be used, so as to remove noises. However, it is often difficult to prepare a large number of samples as many as hundreds of samples and perform gene expression analysis such as RNA-Seq.

In the transcriptome analysis described in this example, a relatively small number of samples (22 lines; i.e., 20 BIL lines and 2 parent lines) were used, so as to detect biomarkers for expression levels indicating the seedling weight. As a result of the transcriptome analysis described in this example, 2 types of known genes; i.e., qLTG3-1 and SG1, with or without a genomic mutation were detected as candidate biomarkers. The results demonstrate that the transcriptome analysis described in this example may enable effective selection of biomarkers for expression levels through analysis of a relatively small number of samples.

### Example 2

In this example, the list of genes selected at high frequency prepared in Example 1 (FIG. 13) was used to calculate the estimated fresh weights of the terrestrial parts of the seedlings.

### 1. Method

Among the 158 genes in the list of genes selected at high frequency prepared in Example 1 (FIG. 13), the fresh weights of the terrestrial parts of the seedlings were estimated based on the gene expression levels by the random forest method (Breiman, L., 2001, Machine Learning 45: 5-32) using the gene expression levels of the top 100 genes in the list and the fresh weights of the terrestrial parts of the seedlings (FIG. 12). In the random forest method, concerning these 100 genes, the expression level data and the fresh weights of the terrestrial parts of the seedlings measured in Example 1 are inputted to prepare estimation model formulae in the form of decision trees, and the estimated values are calculated based on the expression level data of the 100 genes in accordance with the estimation model formulae.

### 2. Results

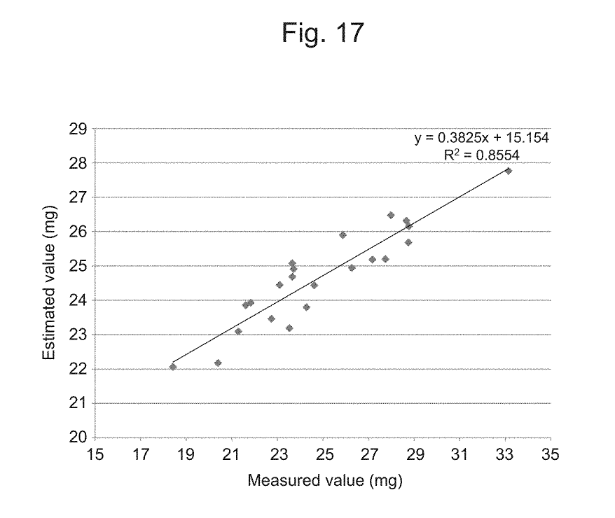

A 5-fold cross validation was repeated 20 times to determine the estimated fresh weights of the terrestrial parts of the seedlings. FIG. 17 shows a chart plotting the measured fresh weights of the terrestrial parts of the seedlings on the horizontal axis and the estimated value determined in accordance with the estimation model formula (the average) on the vertical axis. R² (the adjusted R-square) of the data shown in FIG. 17 was found to be 0.8554. This indicates a very high degree of compatibility. Specifically, the estimation model formulae prepared using the gene expression level data concerning the genes included in the list prepared in Example 1 and the fresh weights of the terrestrial parts of the seedlings would lead to actual data, and explanatory variables (gene expression level data) satisfactorily describe objective variable (the fresh weights of the terrestrial parts of the seedlings).

What is claimed is:

1. An apparatus for transcriptome analysis comprising:
a data set-generator that generates the first to the mth subdata sets (m≥2) by randomly deleting gene expression level data from a plurality of data sets including objective variable data and gene expression level data;
an estimation formula-calculator that applies the method of regression analysis having regularization terms to each of the first to the mth subdata sets to calculate the first to the mth estimation formulae using objective variable data as objective variables and gene expression level data as explanatory variables; and
a list of genes-generator that generates a list of genes corresponding to the gene expression level data included in the first to the mth estimation formulae.

2. The apparatus for transcriptome analysis according to claim 1, wherein the estimation formula-calculator adopts the least absolute shrinkage and selection operator (LASSO) method as the method of regression analysis.

3. The apparatus for transcriptome analysis according to claim 1, wherein the data set-generator generates 1,000 to 20,000 types of subdata sets (m=1,000 to 20,000).

4. The apparatus for transcriptome analysis according to claim 1, wherein the list of genes-generator calculates the appearance probability of gene based on the results of the first to the mth estimation formulae and generates a list of genes in connection with the calculated appearance probability.

5. The apparatus for transcriptome analysis according to claim 1, wherein the list of genes-generator retrieves the annotation information of the genes included in the list from the database storing the annotation information of the genes and generates a list of genes in connection with the retrieved annotation information.

6. The apparatus for transcriptome analysis according to claim 1, which further comprises a estimation model formulae-generator that generates estimation model formulae concerning given objective variables by subjecting a plurality of genes included in the list generated by the list of genes-generator to multiple regression analysis using the objective variable data and the gene expression level data included in the plurality of data sets.

7. A method of transcriptome analysis comprising:
a step of generating a subdata set that generates a subdata set by randomly deleting gene expression level data from a plurality of data sets including objective variable data and gene expression level data;
a step of calculating an estimation formula that applies the method of regularization to the subdata set to calculate an estimation formula using objective variable data as objective variables and gene expression level data as explanatory variables;
a step of gene recording that records genes corresponding to the gene expression level data included in the estimation formula; and
a step of generating a list of genes that repeats the step of generating the subdata set, the step of calculating an estimation formula and the step of gene recording in number of times (m≥2) to generate a list of the recorded genes.

8. The method of transcriptome analysis according to claim 7, wherein the step of calculating an estimation formula adopts the least absolute shrinkage and selection operator (LASSO) method as the method of regularization.

9. The method of transcriptome analysis according to claim 7, wherein the step of generating a subdata set generates 1,000 to 20,000 types of subdata sets (m=1,000 to 20,000).

10. The method of transcriptome analysis according to claim 7, wherein the step of generating a list of genes calculates the appearance probability of gene based on the results of the first to the mth estimation formulae generated through repetition of 1 to m number of times and generates a list of genes in connection with the calculated appearance probability.

11. The method of transcriptome analysis according to claim 7, wherein the step of generating a list of genes retrieves the annotation information of the genes included in the list from the database storing the annotation information of the genes and generates a list of genes in connection with the retrieved annotation information.

12.The method of transcriptome analysis according to claim 7, which further comprises, following the step of generating a list of genes, a step of generating an estimation model formula that generates an estimation model formula concerning given objective variables by subjecting a plurality of genes included in the generated list to multiple regression analysis using the objective variable data and the gene expression level data included in the plurality of data sets.


SEQUENCE LISTING (6 sequences)

SEQ ID NO: 1 - DNA, 20 bp, Artificial Sequence
  Synthetic DNA
gggacgtgataaccgactca

SEQ ID NO: 2 - DNA, 20 bp, Artificial Sequence
  Synthetic DNA
ccccactgtacgttctctcc

SEQ ID NO: 3 - DNA, 20 bp, Artificial Sequence
  Synthetic DNA
gggacgtgataaccgactca

SEQ ID NO: 4 - DNA, 20 bp, Artificial Sequence
  Synthetic DNA
ttcaggtcacctagcccatc

SEQ ID NO: 5 - DNA, 19 bp, Artificial Sequence
  Synthetic DNA
cgaccagctgatctccaag

SEQ ID NO: 6 - DNA, 20 bp, Artificial Sequence
  Synthetic DNA
catttttactggcccttcca